(19) United States
(12) Patent Application Publication
(10) Pub. No.: US 2018/0236118 A1
(43) Pub. Date: Aug. 23, 2018

(54) SILVER NANOPARTICLES IMPREGNATED COVERS FOR ELECTRONIC DEVICES TO COMBAT NOSOCOMIAL INFECTIONS

(71) Applicant: Infection Sciences, LLC, Mobile, AL (US)

(72) Inventors: Sumit Arora, Mobile, AL (US); Om Prakash Jha, Mobile, AL (US); Robert C. Ross, JR., Fairhope, AL (US); J. Mark Swanzy, Mobile, AL (US)

(73) Assignee: Infection Sciences, LLC, Mobile, AL (US)

(21) Appl. No.: 15/902,566

(22) Filed: Feb. 22, 2018

Related U.S. Application Data
(60) Provisional application No. 62/600,486, filed on Feb. 22, 2017.

(51) Int. Cl. A61L 2/238

(52) U.S. Cl. CPC A61L 2/238; B82Y 30/00

(57) ABSTRACT

An anti-microbial covering for use with electronic devices used in a hospital environment is disclosed. A flexible thermoplastic sheet is impregnated during manufacturing with nano-sized sliver particles and wrapped around the electronic device to stop the spread of nosocomial infections. The silver particles vary in shape and size to maximize the anti-microbial effects of each sheet, and each particle is sized to be less than 60 nanometers in diameter. The present invention includes a process for producing each sheet by combining low density polyethylene, linear low density polyethylene, polyphthalamide and other additives with sliver nanoparticles, and then extruding the thermoplastic mixture under heat to form thin impregnated sheets. The liner sheets may then be die-cut to form shaped perforations to facilitate the shaping of each sheet around a targeted electronic device, and cut into convenient sizes for dispensing.

This application claims the benefit of filing priority under 35 U.S.C. § 119 and 37 C.F.R. § 1.78 of the co-pending U.S. Provisional Application Ser. No. 62/600,486 filed Feb. 22, 2017, for a Silver Nanoparticles Impregnated Covers for Electronic Communication Devices to Combat Nosocomial Infections. All information disclosed in that prior pending provisional application is incorporated herein by reference.

### FIELD OF THE INVENTION

The present invention relates generally to materials providing anti-microbial properties. In particular, the invention relates to coverings for electronic devices integrated with an antimicrobial agent or compound. The invention further relates to materials impregnated with silver based substances to combat microbial activity.

### BACKGROUND OF THE INVENTION

Overall incidence of hospital-acquired infections (hereinafter referred to as “HAIs” or simply, “nosocomial infections”) in the USA is on the rise and contributes significantly to morbidity and mortality of patients. HAIs kill more people each year than breast cancer, prostate cancer and AIDS combined in USA making it the fifth leading cause of death in U.S. acute-care hospitals. These infections increase a patient's hospital stay on average from 4.5 to 21.1 days, and kill over 90,000 U.S. patients each year. Centers for Disease Control and prevention (CDC) has estimated that HAIs add an average of $57,000 to a patient's hospital bill and $28 billion to $45 billion added to the nation's healthcare costs each year. According to general medical literature, electronic communication devices (hereinafter “ECDs”) used in hospital are considered as major reservoirs for transmission of nosocomial infections. ECDs are used for communication in every location of the hospital including operating room and intensive care units. More than 70% of the bacteria that cause HAIs are drug resistant organisms. For example, Methicillin-Resistant Staphylococcus Aureus popularly known as “MSRA” is a well-known drug resistant bacterium present in most hospitals that costs lives each year.

Nanotechnology is the convergence of different sciences such as physics, chemistry, biology, material science and medicine, which finds large applications in multiple aspects of research and in everyday life. The availability of new nanomaterials has caused a rapid expansion of the medical arts, often referred to as “nanomedicine,” and are now incorporated into a range of products and technologies. These applications can be in general useful for the management of various microbial infections and in particular for diagnostic and therapeutic uses. Although we live in an era of advanced and innovative technologies for elucidating the underlying mechanism of diseases and creating molecular designs for new drugs, infectious diseases continue to be one of the greatest health challenges worldwide.

The widespread therapeutic use of the antimicrobial chemicals has resulted in bacterial resistance to antibiotics. However, metal nanoparticles have recently become known to be a promising antimicrobial agent that acts on a broad range of target sites on microorganisms, both extracellularly and intracellularly. Moreover, the advances in reducing ions to nanoscale-sized particles have enabled the integration of metal nanoparticles into a large number of materials such as plastics, coating materials, foams and fibers, both natural and synthetic. These nanomaterials have proven their effectiveness for treating infectious diseases, including antibiotic resistance, in vitro as well as in animal models.

Silver is well known for its antimicrobial properties. Silver derives its broad spectrum antimicrobial effect from its ability to bind irreversibly to a variety of nucleophilic groups commonly found in or on the cells of bacteria, viruses, yeast, fungi, and protozoa. Binding to cellular components disrupts the normal reproduction and growth cycle resulting in the death of a cell. Capitalizing on this potent activity, silver in its various compounds and formulations has historically been incorporated into a variety of wound care preparations, such as ointments, hydrogels, hydrocolloids, creams, gels, and lotions. Further, silver nanoparticles are currently being used on the surfaces of various consumer medical products, wound care supplies, and medical treatment supplies, including bandages, dressings, catheters, and sutures, and such usages have proven the safety of silver nanoparticles for human use. After adjusting for the range of effectiveness, the benefits of such infection prevention is expected to be valued at more than $32 billion during the next decade.

According to the medial literature, electronic communication devices or electronic medical devices (hereinafter “ECDs” or “EMDs”) used in hospitals can become major reservoirs for transmission of harmful microorganisms and nosocomial infections. Today, ECDs such as for example, mobile phones, pagers, conference phones, and electronic tablets such as iPads®, have become indispensable accessories of professional and social life among doctors and other health care workers in hospitals. In fact, electronic tablets have quickly become an indispensable device for patient record reviews and updating of patient records during patient exams and procedures. EMDs and ECDs are used for all types of activities and are present in every location of a hospital, including operating rooms and intensive care units. In contrast to the expected benefits of the these devices, EMDs and ECDs are seldom cleaned, but are frequently touched during or after the examination of patients without hand-washing, and have been proven to act as reservoirs for transmission of nosocomial infections. Colonization of potentially pathogenic organisms on EMDs and ECDs has been reported in the literature. Once colonized on the surface of these EMDs and ECDs, infectious microbes can survive for extended periods, unless, these are eliminated by disinfection or sterilization procedure. The United States of America has one of the largest telecommunication networks in the world, and the medical community as with the rest of U.S. society is fully dependent on its telecommunication networks for efficiency. However, despite this efficiency and the known burden of HAIs in hospitals across the U.S., and the growing threat of antibiotic resistant pathogens, no disinfection guidelines have been adopted or issued by the CDC for EMDs or ECDs to reduce nosocomial infections.

While demand for nanoparticles-enhanced products has increased over time, developing techniques for integrating silver nanoparticles into the substrate of products, and in particular EMD or ECD products has remained a challenge. Current approaches result in inefficient use of high value materials, and although there are many approaches to attach nanoparticles to various substrates, those techniques have generally failed to ensure the effectiveness of such nanoparticles or that they remain affixed to such surfaces.

With respect to EMDs or ECDs, these challenges are exacerbated because electronic devices often have a multi-part body which is used to house electronic or other components. Consequently, the smooth contours of the bodies of these devices include various holes, groves, niches, indentations, vents and similar physical features. Such features are typical small, narrow, and difficult to clean, and serve as excellent microorganism reservoirs.

One method of reducing the probability of infection in ECDs is to cover or enclose the device with sanitary or sterile coverings to contain any microbes already present or within a particular ECD to prevent them from contacting a patient or other person who may handle the device. For example, U.S patent application no. 2003/0012371 (“'371”) discloses a cover for a telephone receiver. Although designed to enclose a phone, the '371 invention discloses an open net configuration over the ear and mouth microphones and an open area in the handle portion of the “sock” through which the phone is inserted.

U.S. Pat. No. 8,605,892 (“'892”) discloses a protective instrument cover which appears to cover the entire instrument. It teaches a tube having a continuous wall, an open proximal end, a closed distal end, and sealing means operatively associated with the tube. In another embodiment, '892 additionally discloses a continuous wall containing a reservoir formed therein. The disadvantage of the '892 patent is the cover's material is not integrated with any antimicrobial compound. Therefore, although the cover may prevent bacteria from contacting the instrument, the cover itself may be susceptible to bacterial growth and, thereby become its own microorganism reservoir.

Therefore, what is needed is a material that can serve as a cover or “wrap” to substantially cover the outer surfaces of an electronic device which contains an antimicrobial compound, such as silver nanoparticles, along with a method for impregnating such wraps.

### SUMMARY OF THE INVENTION

The present invention is an anti-microbial covering for an electronic device used in a hospital environment. A flexible thermoplastic sheet is impregnated during manufacturing with nano-sized sliver particles and wrapped around the electronic device to stop the spread of nosocomial infections. The silver particles vary in shape to maximize the anti-microbial effects of each sheet, and each particle is sized to be less than 60 nanometers in diameter. The present invention includes a process for producing each sheet by combining of low density polyethylene, linear low density polyethylene, polyphthalamide and other additives, and sliver nanoparticles, and then extruding the mixture under heat to produce a liner sheet. The liner sheets may then be die-cut to form shaped perforations to facilitate the shaping of each sheet around a targeted electronic device.

Other features and objects and advantages of the present invention will become apparent from a reading of the following description as well as a study of the appended drawings.

### BRIEF DESCRIPTION OF THE DRAWINGS

An anti-microbial covering incorporating the features of the present invention is depicted in the attached drawings which form a portion of the disclosure and wherein:

FIG. 1A is a perspective view of an anti-microbial sheet supported by a folded backing substrate and including perforations and tabs to allow for easy separation of the sheet from the backing substrate;

FIG. 1B is a perspective view of another anti-microbial sheet supported by a folded backing substrate and including concentric perforations;

FIG. 2 a photo micrograph of silver nanoparticles having a rod shape;

FIG. 3 a photo micrograph of silver nanoparticles having an oval shape;

FIG. 4A a photo micrograph of silver nanoparticles having a flower shape;

FIG. 4B a photo micrograph of silver nanoparticles having a prism or triangle shape;

FIG. 5A is a process flow diagram showing an example process to prepare a seed quantity of silver nanoparticles for further use in the process of FIG. 5B;

FIG. 5B is a process flow diagram showing an example process to prepare a quantity of rod shaped silver nanoparticles;

FIG. 6 is a process flow diagram showing a method of making an impregnated anti-microbial sheet;

FIG. 7A is an example of an apparatus for reducing the spread of nosocomial infections using a roller type sheet dispenser;

FIG. 7B is another example of an apparatus for reducing the spread of nosocomial infections using a box type dispenser;

FIG. 8 is an example of an anti-microbial covering in the shape of a bag; and,

FIG. 9 shows an example of an electronic device (tablet) being covered by an anti-microbial covering incorporating the features of the invention.

### DESCRIPTION OF THE PREFERRED EMBODIMENTS

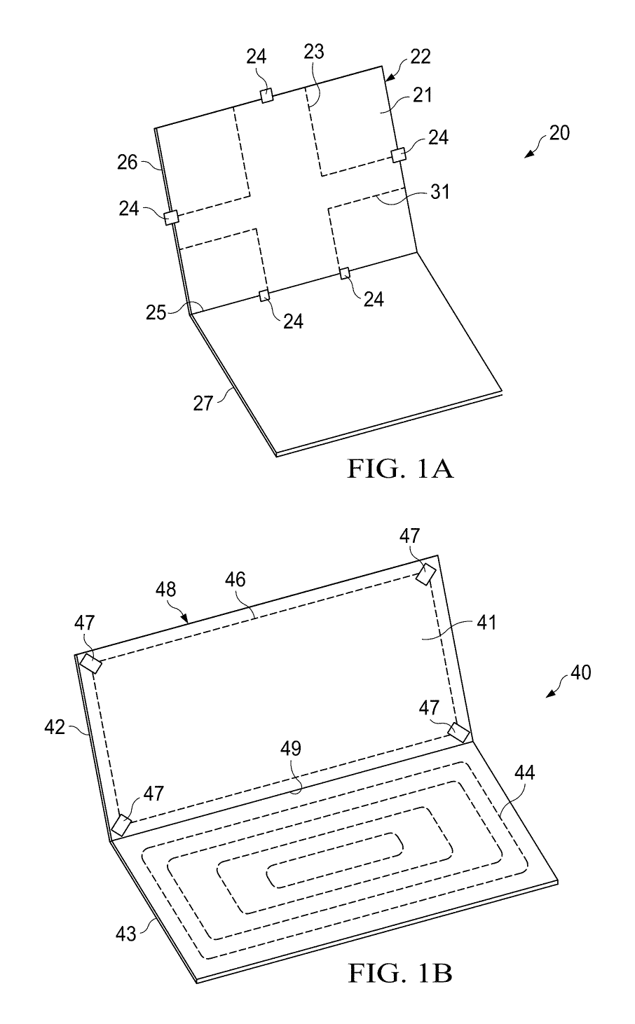

Referring to the drawings for a better understanding of the function and structure of the invention, FIGS. 1A and 1B show example anti-microbial sheets having substrate backings and folded (i.e. configured) to be utilized as a wrap over typical electronic devices used in a hospital environment. The depictions 20,40 each show sheets 20,40 or “wraps” that include a thermoplastic sheet 21,41 impregnated with silver nanoparticles (not shown). Target electronic devices suitable for use in the present invention includes, but are not limited to, smart phones, remote control devices, keyboards, personal computer, tablets, laptop touch-screens, ATM machine monitors, desktop monitors, digital camera screens, GPS navigation screens, mounted touch-screen monitors for factory floors, aviation touch-screen displays, interactive touch-screen displays, interactive white boards (e.g. “smart boards”), iPads®, medical device touch-screens, portable music devices, public display monitors, grocery store self-check-out monitors, touch-screen cash registers, touch-screen coffee machines, touch-screen major appliances, touch-screen media extenders, touch-screen mirrors, touch-screen monitors in vehicles, touch-screen radios, touch-screen soda fountain, touch-screen vending machines, touch-screen tables, touch-screen thermostats, touch-screen televisions, touch-screen voting machines, and touch-screen watches. All of these devices, and more, are utilized in hospital environments. Further, any modern hospital patient room today includes a multiplicity of electronic devices for assisting in the therapy of the patient. For example, patient and treatment rooms can include devices for dispensing medicines, heart monitors, TV control devices, nurse call devices, heart monitors, respiration monitors, and telephones. Moreover, most medical practitioners utilize tablet computing devices that have screens that range in size from 7 to 11 inches to record patient data and issued medical prescriptions.

Each herein described anti-microbial sheet is formed from a base thermoplastic material. The term “thermoplastic,” also known as “thermos softening plastic” is a plastic material, typically comprised of a polymer, which becomes pliable or moldable above a specific temperature and hardens upon cooling. Most thermoplastics have a high molecular weight. The polymer chains associate through intermolecular forces, which weaken rapidly with increased temperature, yielding a viscous liquid. Thus, thermoplastics may be reshaped by heating and are typically used to produce parts by various polymer processing techniques such as injection molding, compression molding, calendaring, and extrusion. The sheets in present invention are principally formed through sheet or balloon extrusion, but various techniques are available for sheet forming of thermoplastics.

Thermoplastic sheets which are suitable for use in the present invention includes, but are not limited to, any light weight or low density polymer, preferably low density polyethylene (“LDPE”). Further, as will be described, LDPE may be combined with linear low density polyethylene (“LLDPE”) to allow for increased resiliency. Additives for such thermoplastic may include polyphthalamide (“PPA”) or polybutene (“PBT”), to increase stiffness, elevate softening temperature, and reduce sensitivity to moisture. The inventors are also utilizing polyvinyl chloride also known as polyvinyl or vinyl, and commonly abbreviated “PVC” as a suitable thermoplastic base. Generally, PVC may replace any other disclosed base thermoplastic utilized in any of the herein described sheet formation processes. The same is true for the unitary use of LLDPE as a thermoplastic.

Each thermoplastic sheet can be integrally attached to a substrate 22,48 with an adhesive component (not shown) and contains a plurality of perforations 23,44, 46, integrated therein such that each sheet is capable of being detached along the perforations utilizing a plurality of tabs 24,47.

Substrates 22,48 suitable for use in the present invention include, but are not limited to, parchment paper, wax paper, polyethylene, polypropylene, polystyrene, polyester, and Glassine, as are known in the industry. Adhesive components suitable for use in the present invention include, but are not limited to, acrylic resin adhesives and the like.

In one preferred embodiment of the present invention, the thermoplastic sheet is constructed of polyethylene such as for example a mixture of the aforementioned LDPE and LLDPE, which is integrated with silver nanoparticles, with each thermoplastic sheet formed to have thickness range of 10 to 1000 microns. Suitable methods of integrating the thermoplastic with silver nanoparticles include, but are not limited to combing particles during extrusion, as will be discussed, and spray coating such particles onto the hardened sheet exterior after cooling.

Referring to FIG. 1A, the thermoplastic sheet 21 is integrally attached to a substrate 22 with an adhesive component (not shown) and contains a plurality of perforations 31 formed therein such that the thermoplastic sheet 21 is capable of being detached along the perforations 31 utilizing a plurality of tabs 24 formed within each thermoplastic sheet 21. The substrate 22 can be bifurcated along a center fold 25 thereby creating a front substrate 26 above the center fold 25 and a back substrate 27 below the fold 25, the perforations 31 being positioned on the front substrate 26 such that a cross-like shape 23 is formed.

For illustration purposes, an electronic device, such as a cell phone, may be placed at the center of the cross shape formed by the perforations 31. The portion of the sheet 21 inside the perforations 31 is lifted from the substrate 22 via tabs 24 and the sheet wrapped over an electronic device, with each arm of the cross shaped portion being folded over the device to cover all portions of its outer surface. The removed portion of the sheet will cling to the outer surface of the device through static attraction, or from any residual adhesive remaining from the substrate 22.

Similarly, FIG. 1B shows another thermoplastic sheet 41 integrally attached to a substrate 48 with an adhesive component (not shown) and contains a plurality of perforations 44,46 integrated therein such that the thermoplastic sheet 41 is capable of being detached along such perforations utilizing a plurality of tabs 47. Substrate 48 may be bifurcated along a center fold 49, thereby creating a front substrate 42 above the center fold 49 and a back substrate 43 below the center fold 49. As may be seen, the perforations 44 supported on the back substrate 43 are configured into concentric shapes so that a user may separate the sheet 41 from the substrate 43 to accommodate various sized devices by selecting and lifting the sheet at an appropriate perforation perimeter. The sheets on the back substrate 43 are shown without lifting tabs, and may be separated from substrate 43 by simple manual manipulation of the combined sheet and substrate. While rectangles with eased corners are shown, it will be understood that any shapes, whether concentric or not, may be formed in the sheets using common die-cutting methods. In operation, a device may be placed on one of the outlined perforation shapes on substrate 43 and a user lifts the appropriate sheet shape in order to cover the entire surface of the device.

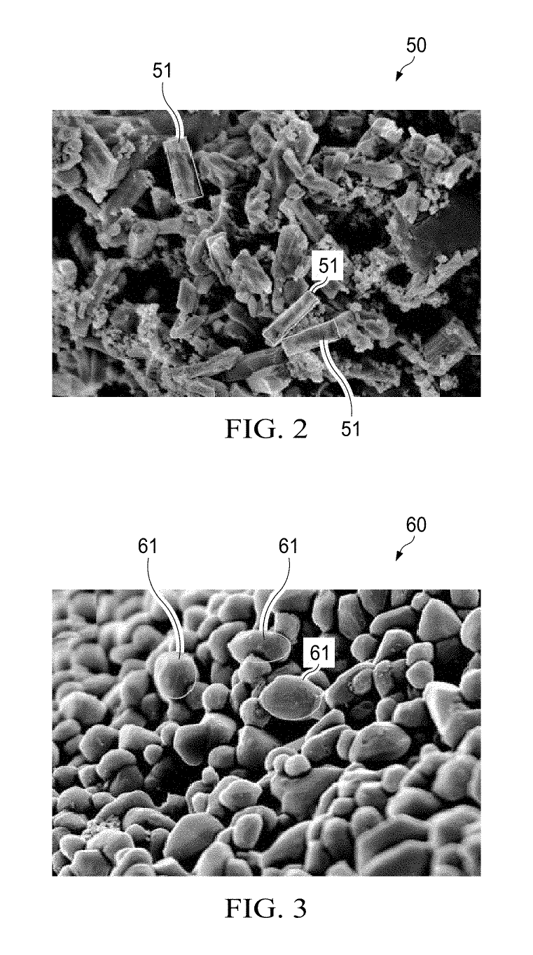

Referring now to FIGS. 2, 3, and 4A-4B, a series of electron micrographs are presented showing the various shapes of silver nanocrystals forming the basis of nanoparticles for impregnation into the present anti-microbial sheets. “Silver nanoparticles” are nanoparticles of silver of between 1 nm and 100 nm in size. Variations in shapes of nanoparticles in general are known to affect the chemical properties of different nanoparticle based substances. The same is true with silver nanoparticles. The extremely large surface area of silver nanoparticles produces many “ligands,” ions or functional molecular groups, which bind to a central metal atom and form a coordination complex. Hence, varying the particle structure of precipitated silver during crystal synthesis results in enhanced anti-microbial effects.

The most common methods for silver nanoparticle synthesis fall under the category of wet chemistry, or the nucleation of particles within a solution. This nucleation occurs when a silver ion complex, usually AgNO₃ or AgClO₄, is reduced to colloidal silver in the presence of a reducing agent. When the concentration increases enough, dissolved metallic silver ions bind together to form a stable surface. The surface is energetically unfavorable when the cluster is small, because the energy gained by decreasing the concentration of dissolved particles is not as high as the energy lost from creating a new surface. When the cluster reaches a certain size, known as the critical radius, it becomes energetically favorable, and thus stable enough to continue to grow. This nucleus then remains in the system and grows as more silver atoms diffuse through the solution and attach to the surface. When the dissolved concentration of atomic silver decreases enough, it is no longer possible for enough atoms to bind together to form a stable nucleus. At this nucleation threshold, new nanoparticles stop being formed, and the remaining dissolved silver is absorbed by diffusion into the growing nanoparticles in the solution. Varying the rate and density of ions through various chemical agents and ambient conditions allows for the shape of the particles to be determined. For example, the attachment of a stabilizing agent will slow and eventually stop the growth of sliver particles. A common capping agent is trisodium citrate and polyvinylpyrrolidone (“PVP”), but others may be used to varying conditions and to control particle size and shape of the silver particles, along with surface properties. Some methods of producing various particle shapes are described below, but a thorough explanation of various chemical techniques used to shape and size silver nanoparticles, and the underlying science, is omitted since such detail is not necessary for a complete and full understanding of the herein described invention.

The inventors have discovered that varying the shapes of silver particles, along with control of their sizes and concentrations, has a strong effect on antimicrobial efficacy. As is known, different shaped silver nanoparticles have different antimicrobial effects on specific bacteria. One explanation for this is that each shape has a different surface to volume ratio and thus each has different high-atom-density facets. These facets act as maximum reactivity sites leading to varying strength in antibacterial activity against bacteria. Based on different cell wall composition of bacteria, Gram-positive and Gram-negative bacteria respond differently to specific shapes. Silver nano-rods and nano-wires are more effective against Gram-positive bacteria, whereas silver nano-prisms (i.e. nano-triangles) are more effective against Gram-negative bacteria as antimicrobial agents. Further, combining different particle shapes greatly increases the total antimicrobial effect of a quantity of silver nanoparticles as an impregnation agent.

Known shapes of silver nanoparticles having antimicrobial properties include: Rod shaped, wire shaped, sphere shaped, oval or ellipsoid shaped, triangle or prism shaped, and flower shaped.

FIG. 2 shows an electronic micrograph 50 of a grouping of silver particles having generally a rod like shape 51 and having a diameter of less than 60 nanometers.

FIG. 3 shows an electronic micrograph 60 of a grouping of silver particles having generally an oval shape 61 and having a diameter of less than 60 nanometers.

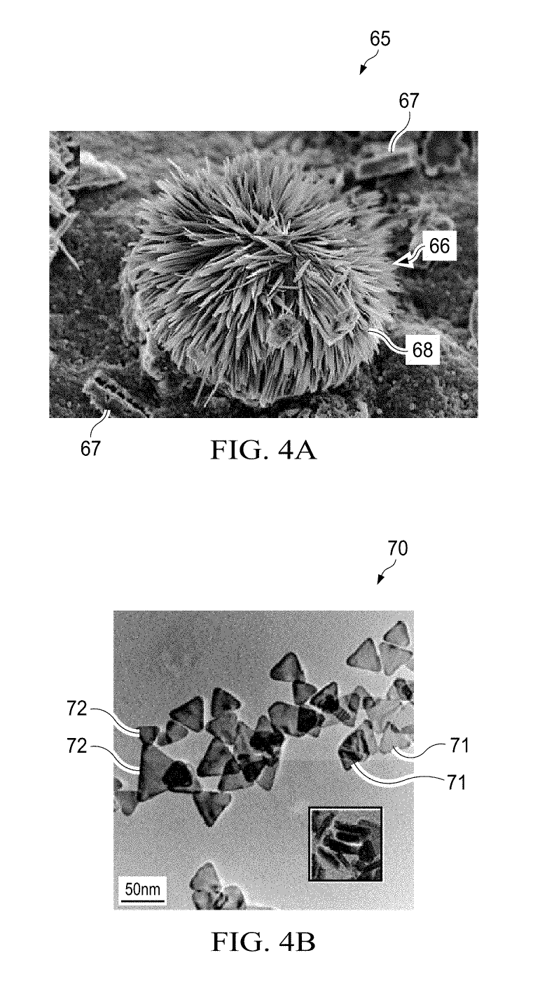

FIG. 4A shows an electronic micrograph 65 of a single silver particle 66 in a varied grouping of different shaped silver particles, such as rods 67. The particle 66 has a generally flowered shape with many particle extensions or “arms” 68 emanating from a central index point. Each arm has a diameter of less than 10 nanometers.

FIG. 4B shows an electronic micrograph 70 of a plurality of silver particles having generally a triangle or “prism” shape 71 in a varied grouping of different sized triangle shaped particles. The triangle shaped particles vary in size 72 of between 20 and 60 nanometers.

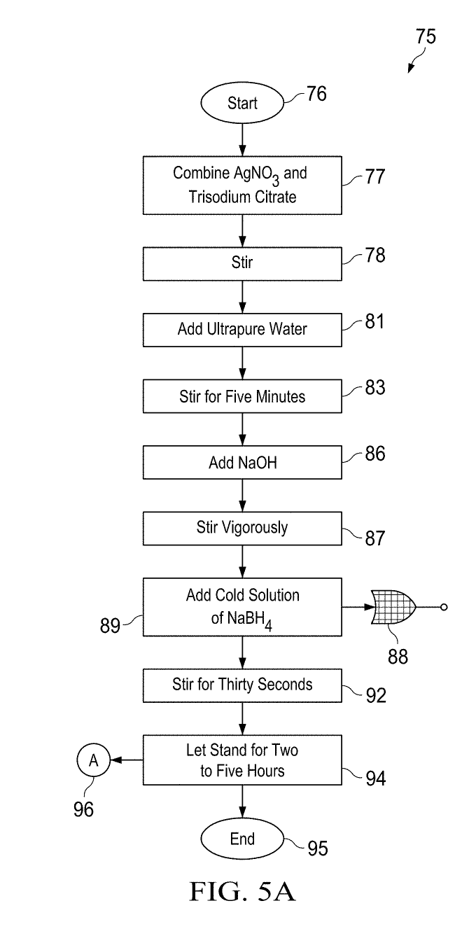

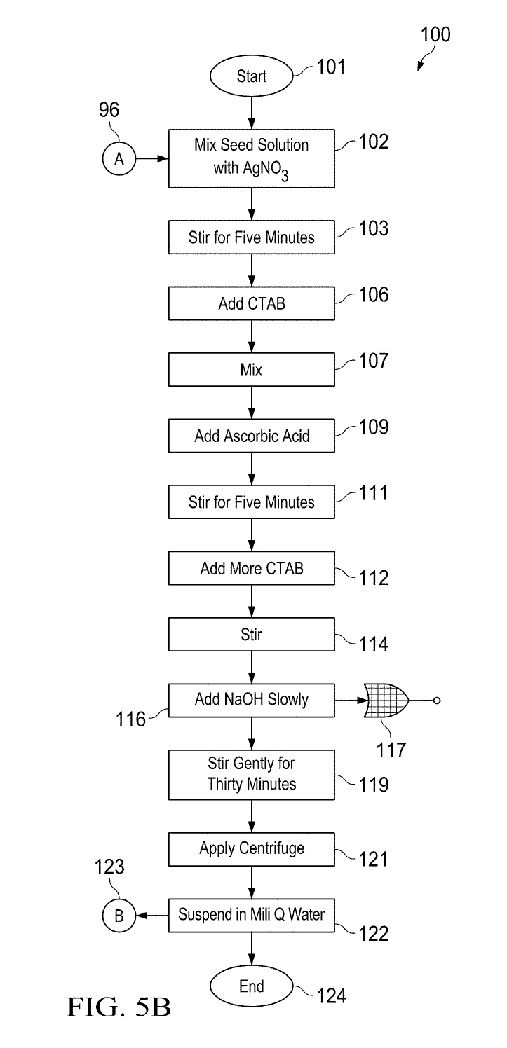

FIGS. 5A-5B show an example process for synthesizing the rod shaped particles shown in FIG. 2. Referring to FIG. 5A, initially a series of chemical steps 75 are taken to produce a “seed” solution of silver particles in a workable volume. As shown, 20 mL of 2.5 mM AgNO₃ solution is combined with 20 mL of 2.5 mM trisodium citrate 77 and stirred 78. One hundred fifty mL of Ultra-pure (e.g. Milli-Q) water is added 81 to the mixture of 77 and stirred for 5 minutes 83. Six mL of 10 mM NaOH solution is added dropwise 86 and stirred vigorously 87, and then 6 mL of ice cold solution of 10 mM NaBH₄ is combined 89 with the solution of 86 while stirring vigorously. The colorless solution should then turn yellow 88 and the yellow solution should be continued to be stirred for 30 seconds 92 and then let stand 94. A seed solution (A) 96 should be available to be use between 2-5 hours after completion of the seed process of 75 and may be used to form specific shapes and sizes of silver nanoparticles with further chemical processing as will be described.

Referring now to FIG. 5B, process 100 discloses a method to produce rod-shaped silver nanoparticles using the seed solution formed in process 75. As shown, 22.5 mL of the seed solution (A) 96 is mixed with 22.5 mL of 10 mM AgNo₃ 102 and stirred well for 5 minutes 103. Twenty mL of 80 mM CTAB (cetyl trimethylammonium bromide) solution is added 106 and mixed well 107. After this, 45.02 mL of 100 mM ascorbic acid solution is added 109 to the mixture of 106 and stirred well for 5 minutes 111. The solution is then enlarged by adding 112 880 mL of 80 mM CTAB with continuous stirring 114. Finally, 9 mL of 1M NaOH solution is added slowly 116 to the above mixture and the solution should turn a yellowish to red color 117. The solution is then continuously stirred gently for an additional 30 minutes 119. The above produces nano rods in solution and a centrifuge is used 121 to separate out the nano-rods. A centrifuge run at 11,000 RPM is typically suitable for such separation. The resulting separated nano rods are left suspended in ultra-pure water 122 until needed for impregnation with a thermoplastic (B) 123 in an extrusion process.

Seed solution A 96 may be further chemically processed to create other shapes and sizes of silver nanoparticles, such as sphere shaped nanoparticles and wire shaped nanoparticles. For example, to produce wire shaped silver nanoparticles, the same process may be used as for forming rod shaped nano particles except that 155.65 mL of silver seed solution is used instead of 22.5 mL in step 102, and 750 mL of 80 mM CTAB is used instead of 880 mL in step 106 of process 100. All other steps are identical in process 100.

To form prism shaped silver nanoparticles, the following steps are satisfactory. As will be observed, no seed solution is utilized in making prism shaped nanoparticles:

Step 1. Combine 885 mL of 0.1 mM AgNo₃ in a beaker and stir.

Step 2. Add 53.4 mL of 30 mM trisodium citrate to the above solution via a dropwise process.

Step 3. Add 53.4 mL of 0.7 mM Polyvinyl Pyrilidone via a dropwise process to above mixture formed in step 2.

Step 4. Add 30% by weight (i.e 2.12 mL) of hydrogen peroxide to the above mixture resulting from step 3 immediately after completion of step 3.

Step 5. Add 884.01 mL of 100 mM sodium borohydrite dropwise to above mixture formed in step 4 and continue stirring.

Step 6. Continue stirring the mixture formed in step 5 for 30 minutes until the solution changes to a brownish red color. A centrifuge may then be utilized on the mixture to separate out the prism nanoparticles.

Sphere shaped silver nanoparticles are formed during the seed production procedure 75 shown in FIG. 5A and hence no additional processing is required except to apply a centrifuge to the seed solution to extract the sphere shaped silver nanoparticles.

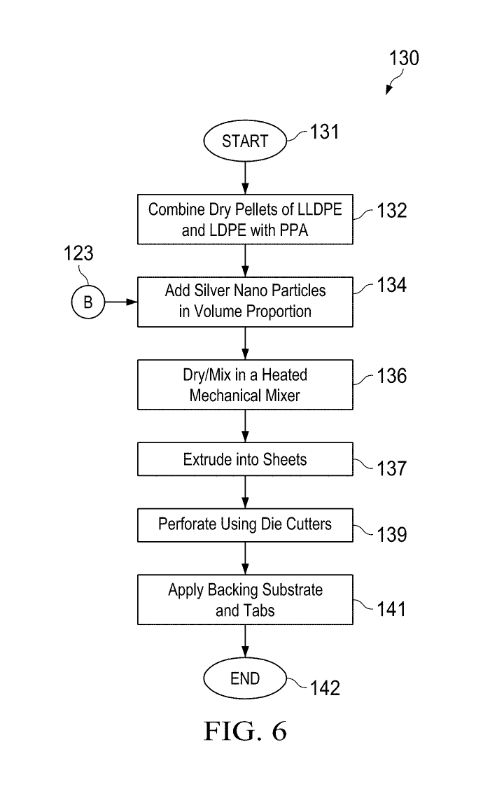

Referring to FIG. 6, process 130 is a suitable process for making an antimicrobial sheet. Pellets of LLDPE and LDPE are combined 132 in a ratio of approximately 80% LDPE and 20% LLDPE by weight. However, a preferred method to make a batch of approximately 10 kg of antimicrobial sheets includes combining of the polyethylene pellets with other additives in dry quantities pursuant to the following component amounts by weight:

| | | |
|---|---|---|
| 1. LLDPE pellets: | 8 | kg |
| 2. LDPE pellets: | 2 | kg |
| 3. Polyphthalamide: | 150 | gm |
| 4. UV protection agents: | 200 | gm |
| 5. Plastic brightener: | 100 | gm |
| 6. Antistatic agent: | 100 | gm |
| Total | 10,550 | gm |
| 7. Silver nanoparticles: | 900 | microgram |
| Total | 10,550.0009 | gm |

The resultant weight percentages are listed below in Table 1.0:

TABLE 1.0

| No. | Component | Weight Percentage |
|---|---|---|
| 1 | LLDPE | 75.82 |
| 2 | LDPE | 18.95 |
| 3 | Polyphthalamide | 1.42 |
| 4 | UV protection agents | 1.89 |
| 5 | Plastic brightener | 0.94 |
| 6 | Antistatic agent | 0.94 |
| 7 | Silver nanoparticles | 0.000009 |

Items 1-6 are combined via dry mixing 132 in a tray or other suitable vessel. The silver nanoparticles (B) 123 from the processes (75,100) in FIGS. 5A and 5B are then combined with the plastic ingredients 134 and dry mixed 136 in the presence of polyethylene glycol to facilitate the removal of water. The silver nanoparticles that are added may include one or more shapes and sizes produced in the above described processes. A preferred concentration ratio of shapes and sizes of silver nanoparticles are 2:1:1:1 for the following shapes, respectively: rods (2); prisms (1); spheres (1); and wires (1). Each of the aforementioned particle shapes may have varying concentrations resulting from the above formation steps. However, in order to meet the above concentration ratios concentrations of each shape must be known and normalized with respect to the concentrations of the other shapes in order to properly combine all of the shapes pursuant to the above stated concentration ratio. So, for example, if a supply source for each of the above preferred shapes was available at a concentration level of 1.0 microgram per mL, and a desired total volume of 5 mL of silver nanoparticles was desired, then in order to meet the above preferred combination ratio, the following quantities shown in Table 2.0 would be needed.

TABLE 2.0

| Concentration | | Source | No. of mL |
|---|---|---|---|
| Ratio | Shape | Concentration | Required |
| 2 | Rods | 1.0 μg/mL | 2 |
| 1 | Prisms | 1.0 μg/mL | 1 |
| 1 | Spheres | 1.0 μg/mL | 1 |
| 1 | Wires | 1.0 μg/mL | 1 |
| | | Total Volume= | 5 mL |

Also, size limitations must be maintained. All silver nanoparticles should be less than or equal to 60 nanometers at their widest diameter, with a preferred range of between 10 and 50 nanometers. While a quantity of 900 micrograms is utilized in the preferred present method for the disclosed quantity, the inventors have seen satisfactory results using a range 500 micrograms to 50 mg of silver nanoparticles in such a process.

The combination of silver nanoparticles with the other thermoplastic ingredients are dry-mixed in a heated mechanical mixer 136 at approximately 300-500 RPM, a temperature of 70° -90° C., and a time duration of 30 minutes. This removes moisture from the silver nanoparticles and thoroughly mixes and impregnates the silver nanoparticles (via absorption) into the thermoplastic. Sheet or balloon extrusion then occurs at 250° C. to produce a thin sheet of silver nanoparticle impregnated thermoplastic 137. The resulting antimicrobial sheet has superior antimicrobial characteristic, is highly flexible, resilient, and transparent. Additional additives may be included to add color to the formed antimicrobial sheets, or increase optical light scattering so that the sheets are translucent. A suitable sheet thickness for the herein described invention is any thickness less than 100 microns. However, by decreasing thickness further, the surface contact area of silver nanoparticles with microorganisms, such as bacteria, increases thereby increasing antimicrobial properties. Hence, a preferred thickness is 30 microns where the tensile strength is sufficient to cover devices such as medical instruments while withstanding regular use by medical personal. Nevertheless, sheets having a thickness of 10-30 microns are possible and would be satisfactory for many medical environments.

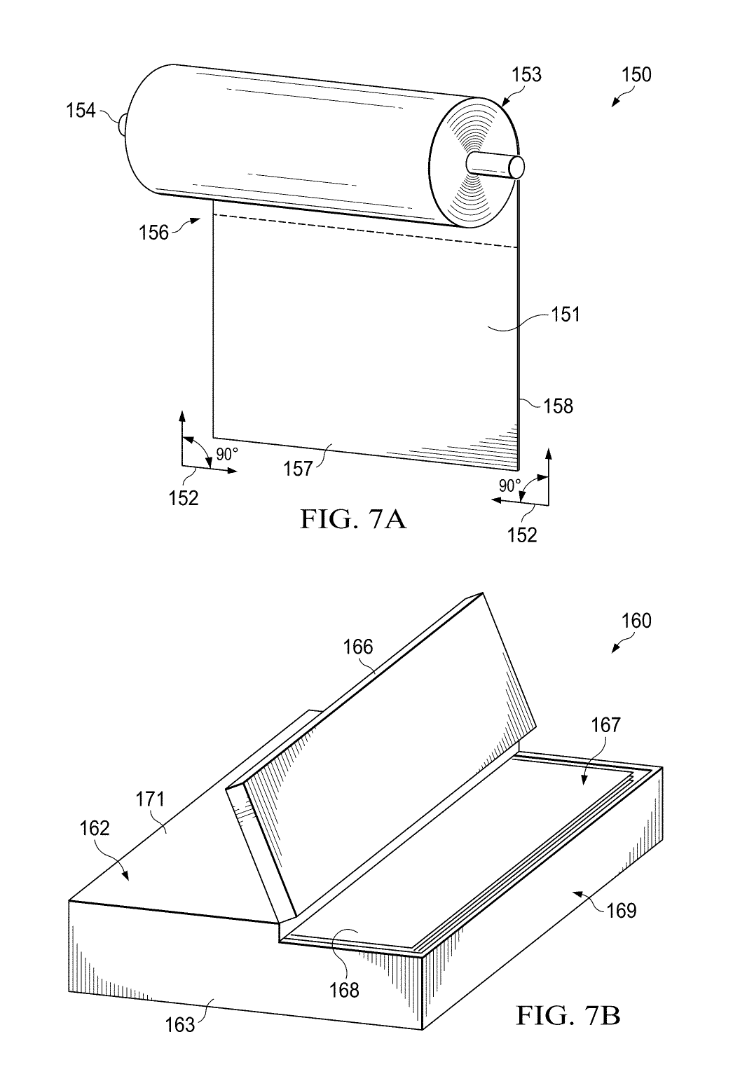

After cooling, a pattern of perforations, or shaped perforations, may be made using die cutters 139. A backing substrate along with tabs may also be added 141 using industry known techniques. Manufactured sheets may be cut into individual sheets and dispensed, or placed on a roller for dispensing by tearing along perforations. For example, FIG. 7A shows a paper towel type dispenser 150 holding a roll 153 of antimicrobial sheets suspended by a roller 154 and positioned in conveniently accessed location in a hospital or clinic environment. Each antimicrobial sheet may include a backing substrate 158 so that a user may grasp the sheet 151 from its lower edge 157 and separate it from roll 153. They may then remove the antimicrobial sheet using manual manipulation or place an electronic device upon the upper surface of the sheet and lift the sheet from its backing to cover the entire surface of the electronic device, as described above in the description for FIGS. 1A-1B.

It is acknowledged that a standard for a minimum level of efficacy against microorganisms is necessary for the current invention. Hence, a minimum inhibitory concentration (“MIC”) of silver nanoparticles solution may be tested against a targeted specific microorganism in a laboratory. Testing may be done with silver nanoparticles in solution against such a targeted microorganism to establish minimum concentration level of silver nanoparticles having varying shapes and sizes. Using those results, a standard of 10 times the minimum effective concentration level may be established as a MIC per square meter of area of a produced antimicrobial sheet. Hence, any organization can target and establish a MIC for its antimicrobial sheets tailored to be used in their medical operations, and a manufacturer can produce and supply such sheets meeting those established minimums. For the process 130 disclosed, a broad MIC of 0.02 to 2 microgram per mL is preferred to achieve a broad spectrum of antimicrobial efficacy in the described antimicrobial sheets produced.

FIG. 7B shows a dispensing box 160 holding a set of stacked, pre-separated sheets 168 accessible via opening 167. A door or lid 166 is biased downward to seal the opening 167 and keep dust or debris from settling on the sheets or inside the dispensing box. The dimensions of the dispensing box determine how many sheets may be stored within it. As may be understood, rear portion 171 may be affixed in a conventional manner to a wall or stand so that the dispenser 160 may be positioned in convenient locations within a hospital or clinic in order for medical personnel to easily access the antimicrobial sheets. A companion platform may also be provided adjacent to the dispensing box (not shown) in order to facilitate the wrapping of the antimicrobial sheet around an electronic device.

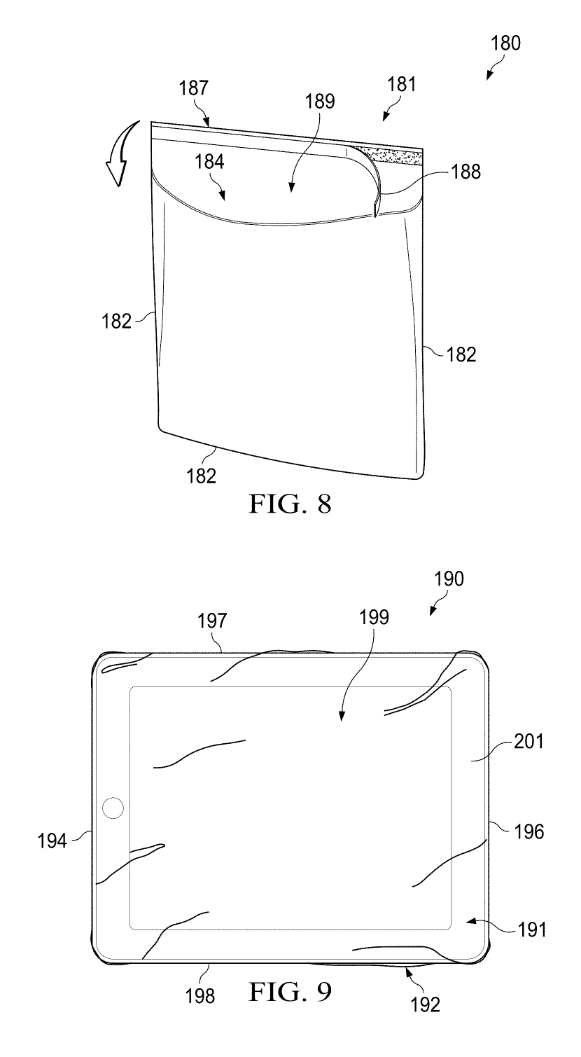

Another convenient shape made from antimicrobial sheets that addresses nosocomial infections is an antimicrobial bag. FIG. 8 shows such a bag 180 that is formed by thermo-sealing a bi-folded antimicrobial sheet at edges 182 to form an opening 184. An elongated edge 181 includes a resealing edge 187 holding an adhesive strip 188, or similar sealing device. Rather than peal and wrap an antimicrobial sheet around an electronic device, a device may simply be placed through opening 184 in direction 189 and the top edge of the bag turned over to engage a lower portion of the bag to seal it. A re-sealable zipper type edge, similar to that used in Ziploc® container bags, may also be used to seal each bag resulting in a bag-type antimicrobial container. Such bag-type antimicrobial containers may be more practical in some environments, especially where response time is limited as in a hospital emergency room. Further, a bag-type antimicrobial container may be simply more convenient for workers to access and utilize. Antimicrobial sheets used to make such a bag are slightly thicker than a nominal single sheet, optimally 40 to 60 microns in thickness, and would vary in size to form bags suitable for enclosing electronic devices of varying sizes and shapes.

Referring now to FIG. 9, an antimicrobial sheet 192 has been placed over an electronic tablet 191 covering around all edges 194, 196, 197, and 198. The antimicrobial sheet exhibits transparent optical properties so that the screen of the tablet 19 may be freely viewed by a user. Further, the inherent thin, flexible nature of any antimicrobial sheet allows for a user to access all buttons or levers necessary on the device without interference with the haptics of the touchscreen. This allows for the normal operation of the tablet by a medical practitioner. Hence, the combination 190 allows for the full functionality of a tablet for hospital use, while interrupting the spread of communicative microorganisms that cause nosocomial infection

While I have shown my invention in one form, it will be obvious to those skilled in the art that it is not so limited but is susceptible of various changes and modifications without departing from the spirit thereof.

Having set forth the nature of the invention, what is claimed is:

1. An anti-microbial covering for an electronic device used in a hospital environment, comprising:
a. a flexible thermoplastic sheet adapted to cover said device;
b. a quantity of nano-sized particles of silver combined with said sheet; and,
c. wherein said sheet combined with said quantity of silver exhibits anti-microbial characteristics.

2. A covering as recited in claim 1, wherein said particles are less than 60 nanometers in diameter.

3. A covering as recited in claim 2, wherein said particles comprise shapes selected from the group consisting of rods, prisms, spheres, wires, flowers, and ovals.

4. A covering as recited in claim 3, wherein said sheet has a thickness of less than or equal to 30 microns.

5. A covering as recited in claim 4, wherein said extruded polyethylene comprises approximately 20 percent by weight of low density polyethylene and approximately 80 percent by weight of linear low density polyethylene.

6. A covering as recited in claim 5, wherein said sheet includes a removable backing substrate supporting said sheet.

7. A covering as recited in claim 6, further comprising shaped perforations to facilitate wrapping said covering over the entire outer surface of said electronic device.

8. A covering as recited in claim 3, wherein said sheet is configured into a sealable bag adapted for receiving said electronic device.

9. A covering as recited in claim 8, wherein said sheet exhibits optical properties selected from the group consisting of transparent, translucent, and colored.

10. A covering as recited in claim 9, wherein said particles comprise equal concentration volumes of prisms, spheres, and wires, and double the concentration volume of rods as compared to any of the other shape concentration volumes.

11. A covering as recited in claim 1, wherein said particles comprise a mixture of rod, sphere, prism, and wire shapes.

12. A covering as recited in claim 11, wherein said thermoplastic sheet comprises extruded polyethylene.

13. A covering as recited in claim 12, wherein said sheet includes a removable backing substrate supporting said sheet.

14. A covering as recited in claim 13, wherein said sheet includes at least one tab elevated from said backing for pulling and separating said sheet from said backing.

15. A covering as recited in claim 1, further comprising a backing substrate supporting said thermoplastic sheet, and wherein said backing substrate is bifurcated along a center fold thereby creating a front substrate portion above said fold and a back substrate portion below said fold, and wherein said sheet on said front substrate comprises a series of perforations arranged thereon to form the shape of a cross.

16. A covering as recited in claim 15, wherein said front portion further includes a plurality of perforated concentric shapes for the selective separation of said sheet from said front substrate to conform to a size of an electronics device intended for wrapping.

17. A covering as recited in claim 16, wherein said sheet exhibits optical properties selected from the group consisting of transparent, translucent, and colored.

18. A process for manufacturing an anti-microbial covering for an electronic device used in a hospital environment, comprising the steps of:
a. preparing a quantity of nano-sized silver particles;
b. preparing a quantity of thermoplastic material;
c. mixing said silver particles with said thermoplastic material; and,
d. extruding said mixture of silver particles with said thermoplastic material under heat to form a flexible anti-microbial sheet.

19. The process as recited in claim 18, where said step of preparing a quantity of nano-sized sliver particles comprises preparing all particles at less than 60 nanometers in diameter.

20. The process as recited in claim 19, where said thermoplastic material comprises polyethylene.

21. The process as recited in claim 20, wherein said thermoplastic material comprises 20 percent by weight of low density polyethylene and approximately 80 percent by weight of linear low density polyethylene.

22. The process as recited in claim 21, wherein said extrusion step comprises extruding a sheet having a thickness of less than or equal to 30 microns.

23. The process as recited in claim 19, wherein said thermoplastic material comprises Polyvinyl chloride.

24. The process as recited in claim 18, wherein said step of preparing a quantity of nano-sized silver particles comprises preparing particles having shapes selected from the group consisting of rods, prisms, spheres, and wires.

25. The process as recited in claim 24, wherein said step of preparing a quantity of nano-sized silver particles comprises combining equal concentration volumes of prisms, spheres, and wires, with double the concentration volume of rods as compared to any of the other single shape concentration volumes.

26. An apparatus for reducing the spread of nosocomial infections by covering electronic devices with an anti-microbial material, comprising:
a. a dispenser located in a healthcare treatment facility;
b. wherein said dispenser holds a quantity of flexible sheets impregnated with nano-sized silver particles;
c. wherein each said flexible sheet comprises thermoplastic made from polyethylene;
d. wherein said nano-sized silver particles have a particle size of less than 60 nanometers; and,
e. wherein each flexible sheet is supported by removable backing material to facilitate the withdrawal of said sheets from said dispenser and for the wrapping of said impregnated sheets over an electronic device present in said healthcare treatment facility.

27. An apparatus as recited in claim 26, wherein each said silver nanoparticle is less than 60 nanometers in diameter, and said silver nanoparticles comprise a mixture of rod, sphere, prism, and wire shapes, and wherein each said sheet has a thickness of less than or equal to 30 microns.

28. An apparatus as recited in claim 27, wherein each said sheet includes a removable backing substrate supporting said same.

29. An apparatus as recited in claim 28, wherein said dispenser comprises a suspended roller for holding said sheets.

30. An apparatus as recited in claim 28, wherein said dispenser comprises an enclosed container having a closable opening for keep dust and debris from settling onto said sheets.

31. An apparatus for reducing the spread of nosocomial infections by covering electronic devices with an anti-microbial material, comprising:
a. a dispenser located in a healthcare treatment facility;
b. wherein said dispenser holds a quantity of sealable bags impregnated with nano-sized silver particles;
c. wherein each bag comprises thermoformed plastic sheets made from polyvinyl chloride;
d. wherein said nano-sized silver particles have a particle size of less than 100 nanometers; and,
e. wherein each said sealable bag includes at least a portion of transparency for viewing the interior of its contents.

32. An apparatus as recited in claim 31, wherein each said silver nanoparticle has a size of between 10 and 40 nanometers in diameter, and wherein said silver nanoparticles comprise a mixture of rod, sphere, prism, and wire shapes, and wherein each said bag has a wall thickness of less than or equal to 40 microns.

33. An apparatus as recited in claim 32, wherein said sheet comprises silver particles having equal concentration volumes of prisms, spheres, and wires, with double the concentration volume of rods as compared to any of the other shape concentration volumes.
